(19) United States
(12) Patent Application Publication
(10) Pub. No.: US 2023/0128112 A1
(43) Pub. Date: Apr. 27, 2023

(54) SECUREMENT DEVICE FOR MEDICAL TUBING

(71) Applicant: Children's Hospital Medical Center of Akron, Akron, OH (US)

(72) Inventor: MOLLY DUDA, AKRON, OH (US)

(21) Appl. No.: 17/973,924

(22) Filed: Oct. 26, 2022

Related U.S. Application Data
(60) Provisional application No. 63/271,961, filed on Oct. 26, 2021.

(51) Int. Cl. A61M 25/02

(52) U.S. Cl. CPC A61M 25/02; A61M 2025/026

(57) ABSTRACT

One or more techniques and/or systems are disclosed for a securement device for medical tubing including a base member having a proximal end and a distal end, wherein the base member is substantially free of adhesive, and a flap operably connected to the base member, wherein the flap and the base member together define an opening configured to accommodate medical tubing. The flap has a proximal end affixed to the base member and a distal end that is selectively detachable from the base member. A fabric retaining member is operably connected to the base member and includes a distal end that is affixed to the base member and a proximal end. A first fastener is operably connected to the fabric retaining member, the first fastener is configured to releasably couple the fabric retaining member with the base member proximate to the distal end of the flap.

### RELATED APPLICATION DATA

This application claims priority to provisional application having serial number 63/271,961, which was filed on Oct. 26, 2021, which is incorporated by reference herein in its entirety.

### BACKGROUND

The use of medical tubing in medical procedures and medical care occurs for patient populations, including adults, pediatrics, and even in the veterinary setting. One example of such tubing, is central lines. Central lines may be at risk for infection and line dislodgement. This could be true for pediatric patients. Diapered patients may have a special securement of central line tubing to keep out of diaper area with potential exposure to bodily fluids. Diapered patients often have IV tubing, EKG leads, and other tubing that comes near or is secured near the diaper area, where bodily fluids reside. Central line dressings may be changed as frequently as weekly or more. There is a need in the art for a device that keeps medical tubing away from less than desired body locations and can have repeat use, is easy to clean, is cost effective, and can be made readily available to the general public.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

Provided herein is one implementation of a securement device for medical tubing. The securement device may comprise a base member that may comprise a proximal end and a distal end, wherein the base member is substantially free of adhesive. The securement device may further comprise a flap operably connected to the base member, wherein the flap and the base member together define an opening configured to accommodate medical tubing. The flap may comprise a proximal end affixed to the base member and a distal end that is selectively detachable from the base member. Additionally, the securement device may comprise a fabric retaining member operably connected to the base member. The fabric retaining member may comprise a distal end that is affixed to the base member and a proximal end. The securement device may further comprise a first fastener operably connected to the fabric retaining member, the first fastener may be configured to releasably couple the fabric retaining member with the base member proximate to the distal end of the flap.

Provided herein is another implementation of a securement device for medical tubing. The securement device may comprise a base member having a proximal end and a distal end that includes a first fastener component, wherein the base member is substantially free of adhesive. The securement device may further comprise a flap operably connected to the base member, wherein the flap and base member together define an opening configured to accommodate medical tubing. The flap may comprise a proximal end affixed to the base member and a distal end that is selectively detachable from the base member. Additionally, the securement device may comprise a fabric retaining member operably connected to the base member. The fabric retaining member may comprise a distal end that is affixed to the base member and a proximal end that includes a first fastener component. The first fastener component of the fabric retaining member may be configured to engage the first fastener component of the base member to form a first fastener, wherein the first fastener may be configured to mutually, releasably couple the fabric retaining member with the base member proximate to the distal end of the flap.

Provided herein is still another implementation of a securement device for medical tubing. The securement device may comprise a base member having a proximal end and a distal end that includes a first fastener component, wherein the base member is substantially free of adhesive and further includes a second fastener component disposed proximate to the first fastener component. The securement device may further comprise a flap operably connected to the base member, the flap and base member together defining an opening configured to accommodate medical tubing. The flap may comprise a proximal end affixed to the base member and a distal end that is selectively detachable from the base member, wherein the flap includes a second fastener component at the distal end of the flap. Additionally, the securement device may comprise a fabric retaining member operably connected to the base member. The fabric retaining member may comprise a distal end that is affixed to the base member and a proximal end that includes a first fastener component. The first fastener component of the fabric retaining member may be configured to engage the first fastener component of the base member to form a first fastener, wherein the first fastener may be configured to mutually, releasably couple the fabric retaining member with the base member proximate to the distal end of the flap. Further, the second fastener component of the flap may be configured to engage the second fastener component of the base member to form a second fastener, wherein the second fastener may be configured to mutually, releasably couple the flap with the base member proximate to the first fastener.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### BRIEF DESCRIPTION OF THE DRAWINGS

What is disclosed herein may take physical form in certain parts and arrangement of parts, and will be described in detail in this specification and illustrated in the accompanying drawings which form a part hereof and wherein:

FIGS. 1A through 1C are component diagrams illustrating a top view of various example implementations of a securement device for securing medical tubing in accordance with this disclosure.

FIGS. 2A through 2C are component diagrams illustrating a top view of one or more portions of the securement devices of FIGS. 1A through 1C.

FIGS. 3A and 3B are component diagrams illustrating a side view of one or more portions of the securement device of FIGS. 1A and 2A configured in a closed and open position.

FIGS. 4A and 4B are component diagrams illustrating a side view of one or more portions of the securement device of FIG. 2C configured in a closed and open position.

FIGS. 5A and 5B are component diagrams illustrating a top view of one or more portions of the securement devices of FIGS. 2C and 4A through 4B configured in different positions.

FIGS. 6A and 6B are component diagrams illustrating various views of the securement device of FIGS. 5A and 5B attached to a garment for use.

FIG. 7 is a flow diagram illustrating an example implementation of an example method for using the securement device of this disclosure to secure medical tubing.

FIG. 8 is a flow diagram illustrating an example implementation of an example method for using the securement device of this disclosure to secure medical tubing.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

Medical tubing in medical procedures and medical care occurs for patient populations, including adults, pediatrics, and even in the veterinary setting. One example of such tubing, is central lines. Central lines may be at risk for infection and line dislodgement. Pediatric patients with central lines are a vulnerable patient population at risk for developing central line associated blood stream infection. There are challenges in securing central lines in pediatric patients. Central line associated blood stream infection such as bacteria, virus, fungi enter the blood stream and can lead to sepsis. Central lines usually have entry points for infection, which could include, intravenous (“IV”) tubing, cap, central line tubing, and the insertion site into the skin (underneath the sterile cover in place).

The tip of the catheter terminates in a large blood vessel right above the heart. This allows for direct access into the bloodstream. Catheter dislodgement occurs when a force is exerted on a line, either on the catheter itself, or the tubing connection. This can cause the line (catheter) to come out. Alternatively, this could lead to catheter breakage or fracture, IV tubing connection being pulled, yanked, stepped on, or even chewed on by the patient. For pediatric patients, tubing can often migrate to the diaper area unintentionally which can lead to exposure to bodily fluids and substances, such as stool or urine, which is not desirable. Examples of medical tubing may include without limitation, catheters, central lines, EKG leads, feeding tubes and other tubing utilized for medical procedures and medical care.

Provided herein is a securement device for medical tubing that orients the position of the medical tubing over the patient’s body, such as over the shoulder, thereby avoiding areas that may less desired for the medical tubing including areas having frequent movement or an area that could encounter bodily fluids. Thus, the securement device of this disclosure provides an inexpensive way to keep a patient’s tubing, such as a central line, secured in a clean location over the body portion that has less frequent body motion or contact with bodily fluid, such as over the patient’s shoulder. As a result, the securement device of this disclosure helps reduce or even prevent infection, dislodgement, and breakage of medical tubing, such as central lines. Additionally, the securement device described herein may prevent tension and pull on medical tubing.

The securement device of this disclosure can be releasably attached to the patient’s clothing, rather than directly to the patient’s skin, to secure the medical tubing without applying adhesive to the skin. In one implementation, the securement device is releasably attachable to a garment of clothing by wrapping the device around a garment of clothing. In another implementation, the securement device is releasably attachable through the fabric of the garment. Thus, the securement devices provided herein may be used with various types of garments including a short sleeve shirt, long sleeved shirt, hospital gown, or onesie. Moreover, the securement device provided herein is more affordable, offers universal fit of patients, and is user friendly for patients, families, and healthcare workers. Further the securement device may be used with pediatric patients, adults, geriatric patients, and pets or animals.

It will be understood by those of skill in the art in view of the present disclosure that the securement device provided herein can be used with any number of articles and medical devices, including, but not limited to: catheters, connector fittings, catheter hubs, catheter adaptors, fluid delivery tubes, and other medical devices or their components, and electrical wires and cables connected to external or implanted electronic devices or sensors. One skilled in the art may also find additional applications for the securement devices disclosed herein aside from use with the medical articles and devices mentioned above. Thus, the illustrations and descriptions of the securement device in connection with the medical articles are merely exemplary of some possible applications of the securement device.

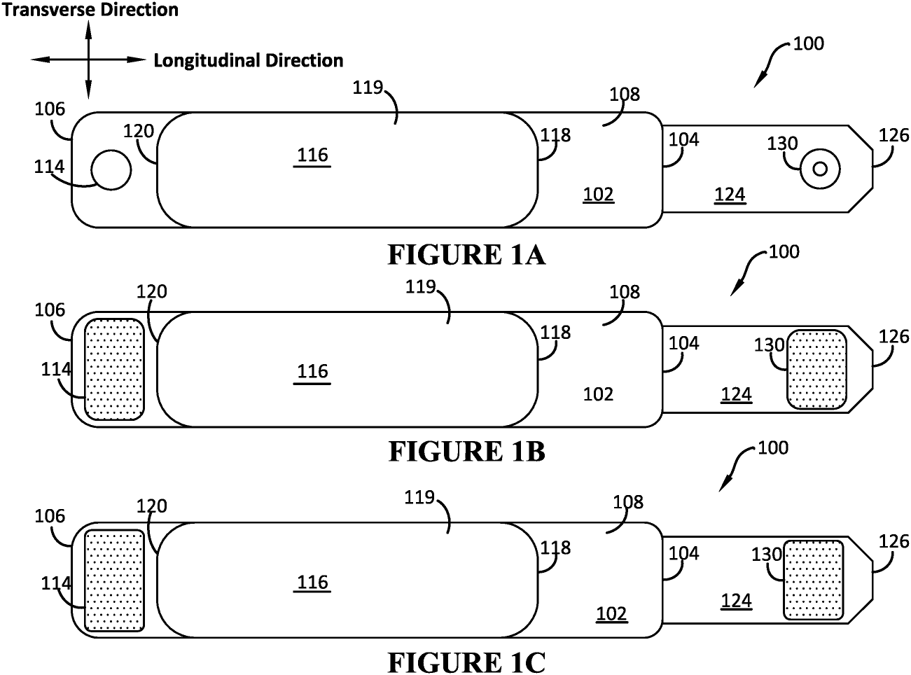

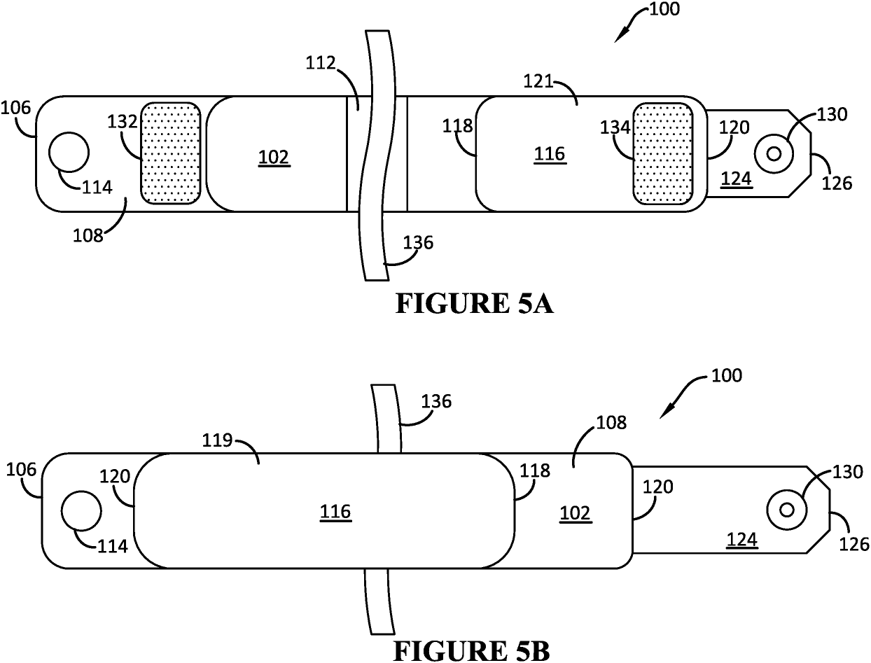

Referring now to FIGS. 1-6, which illustrate a securement device 100 for medical tubing. The securement device 100 may comprise a base member 102 that is substantially free of adhesive; a flap 116 operably connected to the base member 102 with the flap 116 and base member 102 together defining an opening 122 having a size and shape configured to accommodate medical tubing 136 within the opening 122; a fabric retaining member 124 operably connected to the base member 102; and a first fastener operably connected to the fabric retaining member, as described in more detail below. It will be appreciated the securement device 100 disclosed herein can be a single use device or a multiple use device.

The base member 102 may comprise a body that extends between a proximal end 104 and a distal end 106 and is substantially free of adhesive. In some implementations, the base member 102 comprises an elongated body that extends in a longitudinal direction between the proximal end 104 and the distal end 106, the base member 102 being shorter in the transverse direction than in the longitudinal direction. In some implementations, the base member 102 may have a rounded/curved proximal end 104 and/or distal end 106. In some non-limiting examples, the proximal end 104 and/or the distal end 106 may be concave or convex. In the implementations shown in FIGS. 1-5, the base member 102 comprises a rounded proximal end 104 and a rounded distal end 106. In other implementations, the base member 102 may comprise a proximal end 104 and/or distal end 106 that is substantially straight.

The base member 102 may be made of a flexible material that allows the base member 102 to be wrapped or positioned about the fabric of a garment, as described in more detail below. In some implementations, the base member 102 comprises a thin, flexible elongated body that extends in the longitudinal direction between the proximal end 104 and the distal end 106. In some non-limiting examples, the base member 102 is made of silicone.

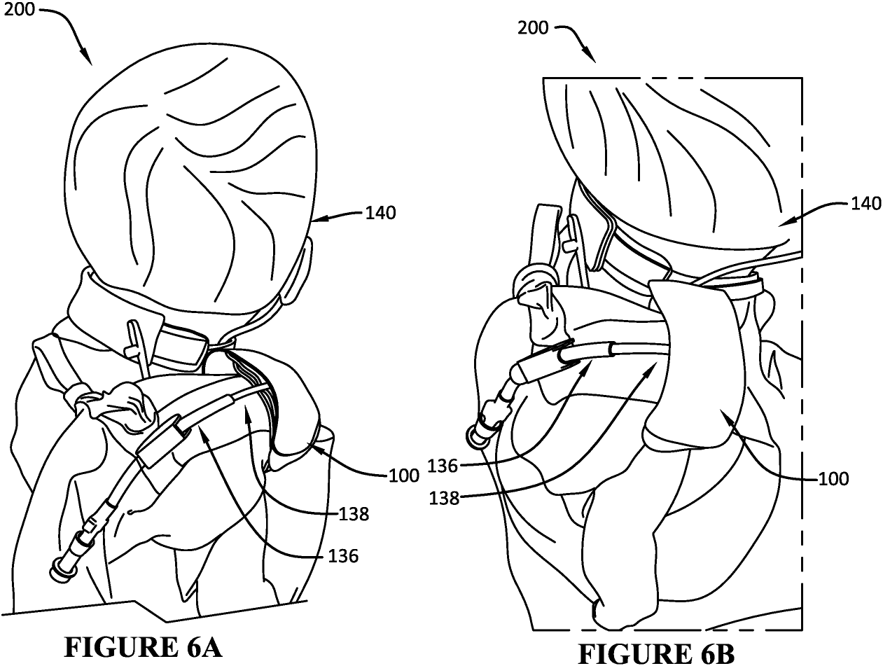

The base member 102 further comprises a first side 108 and a second side 110. FIGS. 6A and 6B show that during use of the securement device 100, the first side 108 is outwardly facing and can be considered the top side while the second side 110 can be considered the bottom side.

The base member 102 may include a channel 112 formed in the first side 108 of the base member 102. In some implementations, the base member 102 may include a raised platform 109 that extends upward from the surface of the first side 108, or top side. In some non-limiting examples, the platform 109 comprises a top side that is substantially flat and includes the channel 112 formed therein, as shown in FIGS. 6A and 6B. In other implementations, the first side 108 of the base member 102 is substantially flat and includes the channel 112 formed therein.

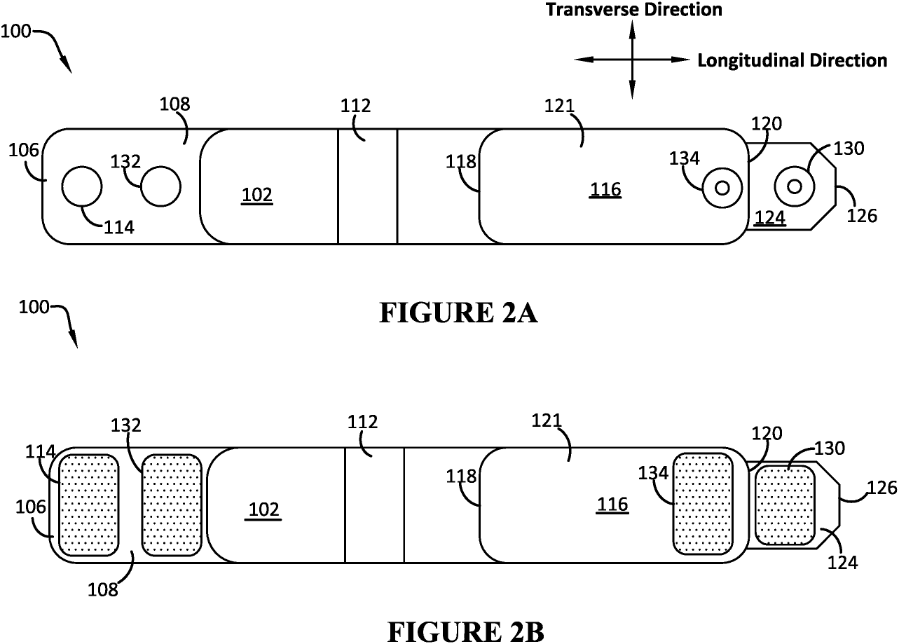

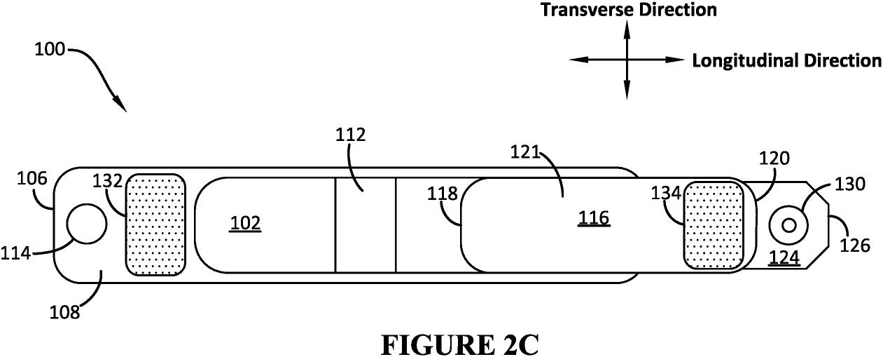

FIGS. 2A-2C show the channel 112 extends in a transverse direction across at least a portion of the width of the base member 102. In some non-limiting examples, the channel 112 extends in the transverse direction across the entire width of the base member 102, as shown in FIGS. 2A-2B. In other non-limiting examples, the channel 112 extends in the transverse direction across most, but not all, of the width of the base member 102, as shown in FIG. 2C. In some implementations, the channel 112 is disposed at or near the midpoint of the base member 102, the midpoint being defined with respect to the longitudinal length of the base member 102. In other implementations, the channel 112 is offset from the midpoint such as, for example, by being positioned closer to the proximal end 104 of the base member 102.

The channel 112 may be configured to accommodate medical tubing 136. The channel 112 may be a variety of different sizes and shapes to accommodate various kinds of medical tubing. In some implementations, the channel 112 may include one or more grooves (not shown) formed in the floor of the channel 112 and adapted to receive the medical tubing within the groove. In some implementations, the channel 112 is lined with compression foam.

The base member 102 may include one or more fastener components that form part of a fastener. In some implementations, the base member 102 may include a first fastener component 114 disposed at or near the distal end 106 of the base member 102. As described in more detail below, the first fastener component 114 is configured to engage a first fastener component 130 that is arranged on a fabric retaining member 124 to form a first fastener 131. In some implementations, the base member 102 may further include a second fastener component 132 disposed proximate to the first fastener component 114 between the first fastener component 114 and the proximal end 104 of the base member 102. As described in more detail below, the second fastener component 132 is configured to engage a second fastener component 134 that is arranged on a flap 116 to form a second fastener 135. Some non-limiting examples of the first fastener components 114, 130 and second fastener components 132, 134 of the respective first and second fasteners 131, 135 include a snap (e.g., 114 in FIGS. 1A, 2A, 3A-3B, 4A-4B, and 5A-5B), a hook-and-loop fastener (e.g., 114 in FIGS. 1B and 2B), a combination thereof (e.g., 114, 132 in FIG. 2C), or some other type of fastener, such as a magnetic fastener (e.g., 114 in FIG. 1C). In some implementations, the first fastener components 114, 130 and/or the second fastener components 132, 134 of the respective first and second fasteners 131, 135 are non-metallic such as, for example, button snaps that are made from plastic. It will be appreciated that a plastic snap avoids dangers associated with wearing a securement device containing metallic elements inside certain medical imaging equipment, for example a MRI machine.

In some implementations, the base member 102 may comprise a flap 116 operably connected to the base member 102. The flap 116 may comprise a body that extends between a proximal end 118 and a distal end 120. In some implementations, the flap 116 comprises an elongated body that extends in a longitudinal direction between the proximal end 118 and the distal end 120, the flap 116 being shorter in a transverse direction than in the longitudinal direction. In some implementations, the flap 116 may have a rounded/curved proximal end 118 and/or distal end 120. In some non-limiting examples, the distal end 120 may be concave or convex. In the implementations shown in FIGS. 1-5, the flap 116 comprises a rounded distal end 120. In other implementations, the flap 116 may comprise a proximal end 118 and/or distal end 120 that is substantially straight.

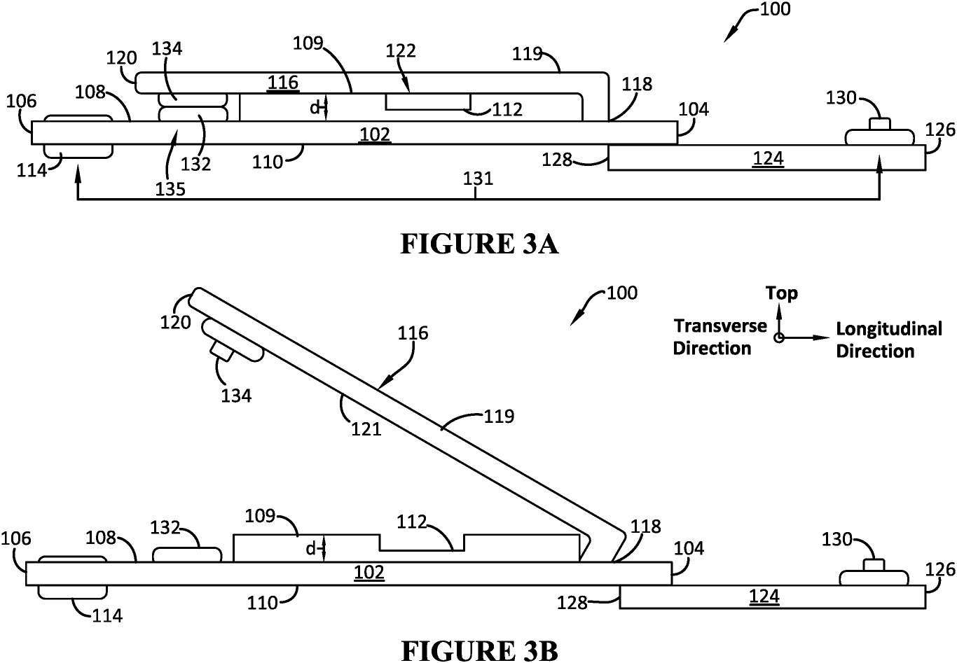

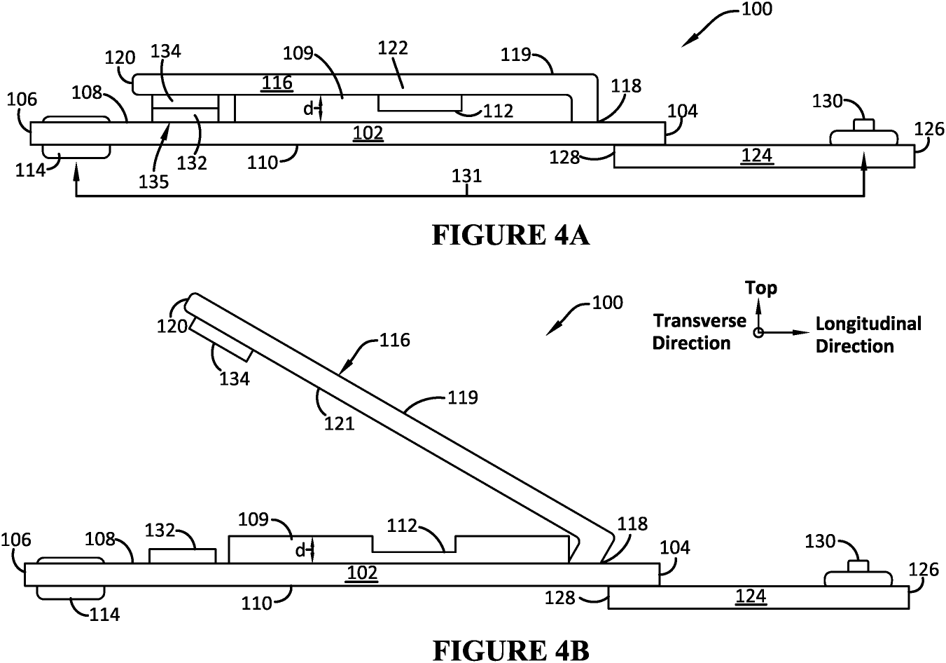

In some implementations, the proximal end 118 of the flap 116 is affixed to the first side 108 of the base member 102 and the distal end 120 is a free end that is selectively attachable to, and selectively detachable (or selectively releasable) from, the first side 108 of the base member 102. As such, the distal end 120 of the flap 116 may be releasably attached to the first side 108 of the base member 102 at a location proximate the first fastener component 114, as shown in FIGS. 3A-3B and 4A-4B. In some implementations, the proximal end 118 of the flap 116 is affixed to the first side 108 of the base member 102 between the raised platform 109 and the proximal end 104 of the base member 102. In other implementations, the proximal end 118 of the flap 116 is affixed to the raised platform 109, preferably to the top side of the platform 109.

The flap 116 further comprises a first side 119 and a second side 121. FIGS. 6A and 6B show that, when the flap 116 is releasably attached to the base member 102, the first side 119 of the flap 116 is outwardly facing and is the top side while the second side 121 of the flap 116, or underside of the flap 116, may be in contact with the raised platform 109 of the base member 102.

The distal end 120 of the flap 116 may be configured to be selectively attachable to, or selectively detachable (or selectively releasable) from, the first side 108 of the base member 102 at a position proximate to the first fastener component 114. In some implementations, the flap 116 may include a second fastener component 134 disposed on the second side 121, or underside, of the flap 116 and disposed at or near the distal end 120 of the flap 116. The second fastener component 134 on the flap 116 may be configured to engage the second fastener component 132 on the first side 108 of the base member 102 to form the second fastener 135. The second fastener 135 is configured to mutually, releasably couple the flap 116 with the base member 102 proximate the first fastener. FIGS. 3A and 4A show example implementations of a securement device 100 in which the flap 116 is in a closed position with the distal end 120 of the flap 116 releasably coupled with the first side 108 of the base member 102 proximate to the first fastener component 114. The distal end 120 of the flap 116 is selectably detachable from the first side of the base member 102 by pulling upward on the distal end 120 of the flap 116 to disengage the second fastener component 134 on the flap 116 from the second fastener component 132 on the base member 102. FIGS. 3B and 4B show example implementations of a securement device 100 in which the flap 116 is in an open position with the distal end 120 of the flap 116 selectively detached from the first side 108 of the base member 102. In some non-limiting examples, the second fastener 135 can comprise a variety of fasteners including, but not limited to, a snap, a hook-and-loop fastener, a magnet, a combination thereof, or some other type of fastener.

In some implementations, the flap 116 and the base member 102 together define an opening 122 that is configured to accommodate medical tubing 136. FIGS. 3A and 4A show the flap 116 in a closed position with the flap 116 releasably coupled with the base member 102 and disposed over the channel 112 in the base member 102. In these implementations, the flap 116 forms a roof over the channel 112 thereby defining the opening 122. In some non-limiting examples, the opening 122 may be configured to hold and removably secure medical tubing therein during use of the securement device 100. In some implementations, the flap 116 may be at least partly supported by the platform 109 when the flap 116 is releasably attached to the base member 102 (e.g., the flap is in the closed position). In these implementations, at least a portion of the second side 121, or underside, of the flap 116 is in contact with the top side of the platform 109, as shown in FIGS. 3A and 4A.

In some implementations, the platform 109 may extend outwardly from the first side 108 of the base member 102 at least a minimum threshold distance, d, sufficient to provide space for the second fastener 135 to be disposed between the base member 102 and the flap 116 and to releasably couple the base member 102 with the flap 116 without obstructing, or preventing, the second side 121 of the flap 116 from contacting the top side of the platform 109. It will be appreciated that these implementations comprise a base member 102 with a platform 109 that is configured to provide support for a flap 116 thereby providing stability to the flap 116 extending over the channel 112 and facilitating engagement between the second fastener components 132, 134 to form the second fastener 135 which mutually, releasably couples the flap 116 with the base member 102.

In some implementations, the distal end 120 of the flap 116 is configured to be selectively releasable, or selectively detachable, from the first side 108 of the base member 102 proximate to the first fastener component 114. The flap 116 is selectively detachable from the base member 102 by pulling upward on the distal end 120 of the flap 116 to disengage the second fastener component 134 on the flap 116 from the second fastener component 132 on the base member 102. It will be appreciated that the flap 116, once selectively detached from the base member 102, may be increasingly opened by moving the distal end 120 of the flap 116 in a direction away from the first fastener 131 and the associated first fastener components 114, 130 and in a direction towards the proximal end 118 of the flap 116. In this way, the flap 116 is configured to transition from a closed position (e.g., the flap 116 is releasably coupled to the base member 102) to an open position (e.g., the flap 116 is selectively detached from the base member 102) without obstructing access to the first fastener 131 and the associated first fastener components 114, 130 because the flap 116 is opened by moving the flap 116 in a direction away from the first fastener 131 and the associated first fastener components 114, 130.

In these implementations, the proximal end 118 of the flap 116 is affixed to the base member 102 in a manner that facilitates movement of the flap 116 relative to the base member 102 about the connection between the proximal end 118 of the flap 116 and the first side 108 of the base member 102. In some non-limiting examples, the connection between the proximal end 118 of the flap 116 and the base member 102 can provide for hinged-type movement of the flap 116 relative to the base member 102.

The securement device 100 further comprises a fabric retaining member 124 operably connected to the proximal end 104 of the base member 102. The fabric retaining member 124 may comprise a body that extends between a proximal end 126 and a distal end 128. In some implementations, the fabric retaining member 124 comprises an elongated body that extends in a longitudinal direction between the proximal end 126 and the distal end 128, the fabric retaining member 124 being shorter in the transverse direction than in the longitudinal direction. In some implementations, the fabric retaining member 124 may have a rounded/curved proximal end 126. The distal end 128 of the fabric retaining member 124 may be affixed at or near the proximal end 104 of the base member 102. In some implementations, the fabric retaining member 124 may comprise a strap or band. In some non-limiting examples, the fabric retaining member 124 is an elastic strap or band.

The fabric retaining member 124 may include a first fastener component 130 at or near its proximal end 126. The first fastener component 130 is configured to engage the first fastener component 114 on the base member 102 to form a first fastener 131. The first fastener 131 is configured to mutually, releasably couple the proximal end 126 of the fabric retaining member 124 with the distal end 106 of the base member 102.

Some non-limiting examples of the first fastener component 130 of the first fastener 131 include a snap (e.g., 130 in FIGS. 1A, 2A, 3A-3B, 4A-4B, and 5A-5B), a hook-and-loop fastener (e.g., 130 in FIGS. 1B and 2B), a combination thereof, or some other type of fastener, such as a magnetic fastener (e.g., 130 in FIG. 1C). In some implementations, the first fastener components 114, 130 are non-metallic such as, for example, button snaps made from plastic. It will be appreciated that a plastic snap avoids dangers associated with wearing a securement device containing metallic elements inside certain medical imaging equipment, for example a MRI machine. It will be further appreciated that the first fastener 131 is configured to be quickly and easily unfastened/released to provide for quick removal of the securement device 100.

In some implementations, the fabric retaining member 124 is made of flexible, stretchable material to allow the size of the fabric retaining member 124 to be adjusted and to allow the fabric retaining member 124 to be wrapped or positioned about the fabric of a garment 138 with minimal bunching of the fabric. The longitudinal length (i.e., size) of the fabric retaining member 124 may be increased by pulling on the proximal end 126 of the fabric retaining member 124 to stretch the fabric retaining member 124 to a length sufficient to encircle the fabric of the garment 138 to releasably attach the securement device 100 to the garment 138 rather than directly to the patient’s body. In some non-limiting examples, the fabric retaining member 124 may be a flexible, stretchable band or strap. In some non-limiting examples, the fabric retaining member 124 is made of a flexible, stretchable material including, but not limited to, a flexible elastomeric material. In other implementations the fabric retaining member 124 is an adjustable strap comprising various preset configurations. It will be appreciated the fabric retaining member 124 may be wrapped or positioned about garments of varying size thereby providing a one-size fits all securement device 100 with minimal bunching of the fabric.

FIGS. 6A and 6B illustrate an example implementation 200 of a securement device 100 releasably attached to a patient’s garment 138 with the securement device 100 being used to secure a patient’s care lines, such as medical tubing 136, within the securement device 100. It will be appreciated that when the securement device 100 is releasably attached to the patient’s garment 138 with the medical tubing 136 secured within the securement device 100, the first fastener 131 and the closed flap 116 are outwardly facing and readily accessible and manipulatable by being generally disposed on a top side of the securement device 100. It will be further appreciated that the securement device 100 is releasably attached to the patient’s garment 138 at a desired location to orient the medical tubing 136 over the patient’s body. By way of example and not limitation, in some implementations the securement device 100 may be releasably attached to the patient’s garment 138 near the neck and shoulder region to orient the medical tubing 136, such as a catheter, over the patient’s shoulder to avoid the diaper area and prevent central line associated blood stream infection (CLABSI). But, it will be appreciated that the securement device 100 can be used for similar securement purposes by being releasably attached to other locations of a patient’s garments or fabrics to orient the medical tubing 136 over the patient’s body. As such, the securement device 100 is not constrained to use only in the neck and shoulder region. It is to be appreciated that one advantage of releasably attaching the securement device 100 in the neck and shoulder region is that the securement device 100 does not need frequent cleaning and/or replacement, yet the securement device 100 still can be easily cleaned and/or replaced when desired.

The securement device 100 of this disclosure provides several advantages which will be readily appreciated. First, it will be appreciated that by releasably attaching the securement device 100 to the patient’s garment 138, rather than to the patient’s body 140 (e.g., skin) using adhesive, the securement device 100 not only secures and stabilizes the patient’s care lines, such as medical tubing 136, in a beneficial position near the shoulder and neck region away from contamination, but additionally avoids applying adhesive products to the patient’s skin thereby avoiding a pressure injury, dermatitis, and/or skin abrasions. The securement device 100 of this disclosure advantageously addresses the need for catheter stabilization and increases patient safety by preventing tension and pull on central line catheters and tubing which can lead to dislodgment, breakage, and infection. It will be appreciated that the securement device 100 of this disclosure is more affordable and user friendly and is adjustable thereby providing a universal fit. It is to be appreciated that the securement device 100 can be more readily/quickly unfastened and removed since the first and second fasteners 131 and 135 are each operably disposed on the first side 108 (e.g., top side) of the base member 102 proximate to each other with both the first and second fasteners 131 and 135 being oriented outwardly (i.e., outwardly facing) to increase visibility and access, thereby allowing rapid interaction.

Another aspect of this disclosure comprises a method of using the securement device 100 provided herein to secure a patient’s care lines, such as medical tubing, by releasably attaching the securement device 100 to a patient’s garment rather than directly to the patient’s skin. In some implementations, the securement device 100 is configured to attach to the patient’s clothing to securely stabilize the medical tubing without using adhesive. In other implementations, the securement device is configured to attach through the patient’s clothing to securely stabilize the patient’s medical tubing within the securement device. Providing two different securement options, as described in greater detail below, provides flexibility and adaptability to accommodate both long sleeved and short sleeved garments.

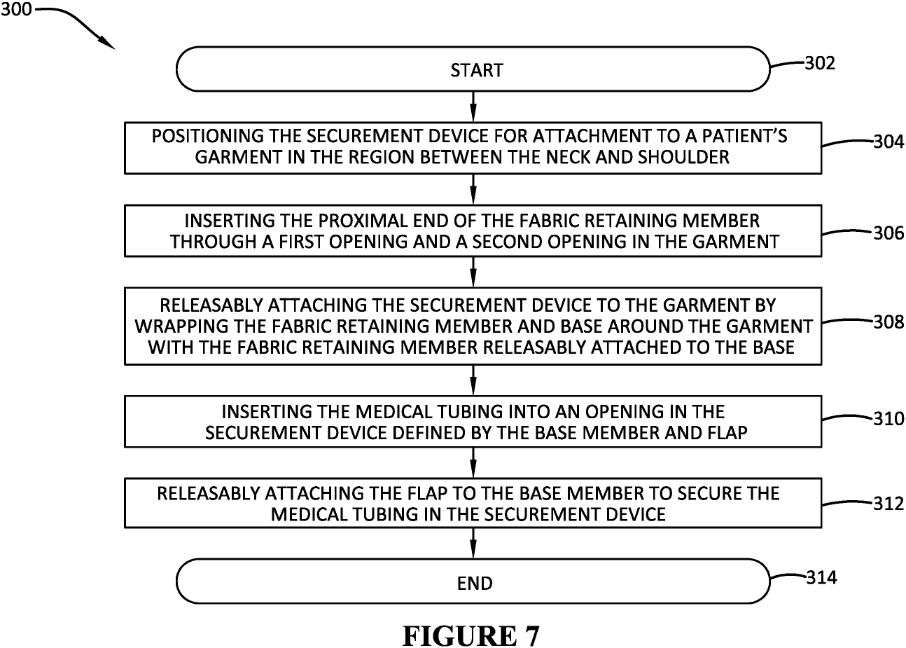

A method may be devised for using the securement device described herein. FIG. 7 is a flow diagram illustrating an example implementation of an exemplary method 300 for using a securement device with a short sleeve garment such as a t-shirt, hospital gown, or onesie. The exemplary method 300 begins at 302. At 304, the securement device can be positioned over the patient’s garment in the region between a first opening and a second opening in the garment, such as between the neck opening and shoulder opening of the garment. In this exemplary method, at 306, the proximal end of the fabric retaining member can be inserted through the first opening (e.g., neck hole opening) and through the second opening (e.g., armhole opening) of the patient’s garment.

In this exemplary method, at 308, the securement device can be releasably attached to the patient’s garment by selectively attaching the fabric retaining member to the base member. In some implementations, a first fastener component disposed at or near the proximal end of the fabric retaining member selectively engages a corresponding first fastener component disposed at or near the distal end of the base member to form a first fastener that mutually, releasably couples the proximal end of the fabric retaining member with the distal end of the base member. That is, for example, the fabric retaining member and base member are wrapped around the fabric of the patient’s garment with the fabric retaining member releasably coupled with the base member with minimal bunching of the fabric. It will be appreciated that the fabric retaining member may comprise a flexible, stretchable strap or band that extends through the neck opening and the armhole opening of the patient’s garment and is configured to stretch as needed to accommodate varying sizes.

In this exemplary method, at 310, medical tubing can be inserted into an opening in the securement device defined by the base member and flap. In some implementations, the flap can be opened by selectively detaching the distal end of the flap from the first side of the base member. In one implementation, the flap can be selectively detachable, or selectively releasable, from the base member by pulling upward on the distal end of the flap to disengage the second fastener component on the flap from the second fastener component on the base member thereby exposing the channel in the base member for accommodating medical tubing.

In this exemplary method, at 312, the flap can be releasably attached to the base member to secure the medical tubing within the opening in the securement device. In some implementations, the flap can be closed by selectively attaching the distal end of the flap to the first side of the base member. In one implementation, the flap can be selectively attachable, or releasably attachable, to the base member by engaging the second fastener component on the flap with the second fastener component on the base member thereby forming the second fastener which mutually, releasably couples the flap with the base member. Having attached the securement device to the patient’s garment with the medical tubing secured within the securement device, the exemplary method 300 ends at 314.

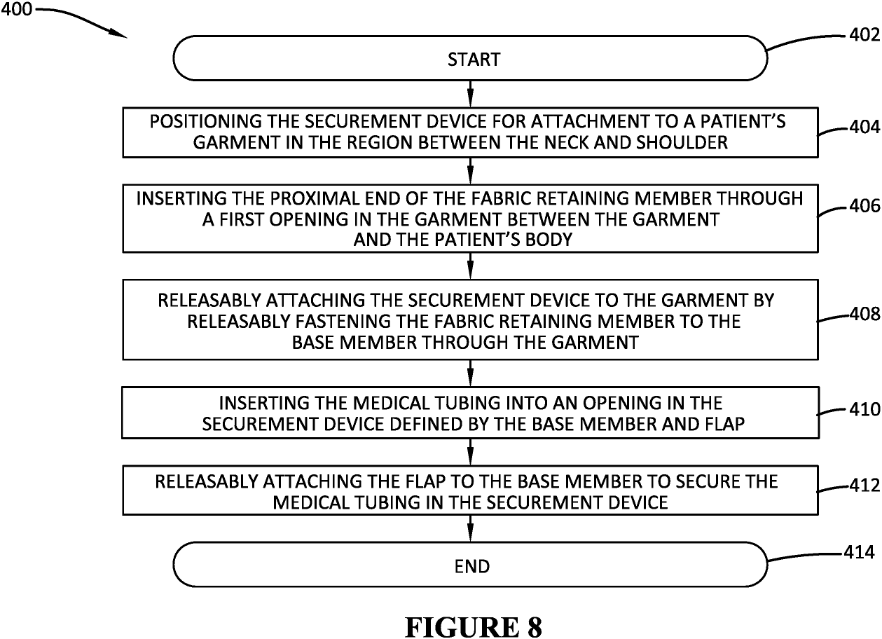

Another method may be devised for using the securement device described herein. FIG. 8 is a flow diagram illustrating an example implementation of an exemplary method 400 for using a securement device with a long-sleeve garment. The exemplary method 400 begins at 402. At 404, the securement device can be positioned over the patient’s garment, for example, in the region between the neck and shoulder. In this exemplary method, at 406, the proximal end of the fabric retaining member can be inserted through a first opening (e.g., neck hole opening) of the patient’s garment such that the fabric retaining member is positioned between the patient’s body (e.g., skin) and the garment. That is, in this example, the long-sleeve garment prevents insertion of the fabric retaining member through both the neck hole opening and the arm hole opening.

In this exemplary method, at 408, the securement device can be releasably attached to the patient’s garment by selectively attaching, or releasably fastening, the fabric retaining member to the base member through the garment or fabric. In some implementations, a first fastener component disposed at or near the proximal end of the fabric retaining member selectively engages a corresponding first fastener component arranged at or near the distal end of the base member to form a first fastener that mutually, releasably couples the proximal end of the fabric retaining member with the distal end of the base member. In some non-limiting examples, to avoid damage to the garment disposed between the base member and the fabric retaining member, the first fastener components comprise magnets. In other non-limiting examples, the first fastener components may comprise a long stud or post that is configured to pass through the weave of a thicker garment and secure to the other fastener component. That is, for example, the fabric retaining member and base member are partially wrapped around the fabric of the patient’s garment with the fabric retaining member releasably coupled to the base member using magnetism. It will be appreciated that the fabric retaining member may comprise a flexible, stretchable strap or band that extends through the neck opening of the patient’s garment and is configured to stretch as needed to accommodate varying sizes.

In this exemplary method, at 410, medical tubing can be inserted into an opening in the securement device defined by the base member and flap. In some implementations, the flap can be opened by selectively detaching the distal end of the flap from the first side of the base member. In one implementation, the flap can be selectively detachable, or selectively releasable, from the base member by pulling upward on the distal end of the flap to disengage the second fastener component on the flap from the second fastener component on the base member thereby exposing the channel in the base member for accommodating medical tubing.

In this exemplary method, at 412, the flap can be releasably attached to the base member to secure the medical tubing within the opening in the securement device. In some implementations, the flap can be closed by selectively attaching the distal end of the flap to the first side of the base member. In one implementation, the flap can be selectively attachable, or releasably attachable, to the base member by engaging the second fastener component on the flap with the second fastener component on the base member thereby forming the second fastener which mutually, releasably couples the flap with the base member. Having attached the securement device to the patient’s garment with the medical tubing secured within the securement device, the exemplary method 400 ends at 414.

The word “exemplary” is used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. Of course, those skilled in the art will recognize many modifications may be made to this configuration without departing from the scope or spirit of the claimed subject matter.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure.

In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

The implementations have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

What is claimed is:

1. A securement device for medical tubing, comprising:
a base member comprising a proximal end and a distal end, wherein the base member is substantially free of adhesive;
a flap operably connected to the base member, wherein the flap and base member together define an opening configured to accommodate medical tubing, the flap comprising a proximal end affixed to the base member and a distal end that is selectively detachable from the base member;
a fabric retaining member operably connected to the base member, the fabric retaining member comprising a distal end that is affixed to the base member and a proximal end; and
a first fastener operably connected to the fabric retaining member, the first fastener configured to releasably couple the fabric retaining member with the base member proximate to the distal end of the flap.

2. The securement device of claim 1, wherein the first fastener comprises:
a first fastener component disposed at the distal end of the base member; and
another first fastener component disposed on the fabric retaining member and configured to engage the first fastener component disposed at the distal end of the base member to mutually, releasably couple the fabric retaining member with the base member.

3. The securement device of claim 2, wherein the first fastener comprises a snap, a hook-and-loop fastener, a magnet, or a combination thereof.

4. The securement device of claim 1, wherein the first fastener and the flap are outwardly facing.

5. The securement device of claim 1, wherein the fabric retaining member is made of a stretchable material that provides for adjusting a size of the fabric retaining member.

6. The securement device of claim 5, wherein the fabric retaining member is an elastic band.

7. The securement device of claim 1, further comprising a second fastener operably connected to the flap, the second fastener configured to releasably couple the flap with the base member proximate to the first fastener.

8. The securement device of claim 7, wherein the second fastener comprises:
a second fastener component disposed on the base member; and
another second fastener component disposed at the distal end of the flap and configured to engage the second fastener component disposed on the base member to mutually, releasably couple the flap with the base member.

9. The securement device of claim 7, wherein the second fastener comprises a snap, a hook-and-loop fastener, a magnet, or a combination thereof.

10. The securement device of claim 7, wherein the first fastener and/or the second fastener is non-metallic.

11. The securement device of claim 1, wherein the flap is configured to be selectively detached from the base member and moved to an open position by moving the distal end of the flap in a direction away from the first fastener.

12. The securement device of claim 1, wherein the base member further includes a channel that is covered by the flap to define the opening.

13. A securement device for medical tubing, comprising:
a base member comprising a proximal end and a distal end that includes a first fastener component, wherein the base member is substantially free of adhesive;
a flap operably connected to the base member, wherein the flap and base member together define an opening configured to accommodate medical tubing, the flap comprising a proximal end affixed to the base member and a distal end that is selectively detachable from the base member; and
a fabric retaining member operably connected to the base member, the fabric retaining member comprising a distal end that is affixed to the base member and a proximal end that includes a first fastener component,
wherein the first fastener component of the fabric retaining member is configured to engage the first fastener component of the base member to form a first fastener, and wherein the first fastener is configured to mutually, releasably couple the fabric retaining member with the base member proximate to the distal end of the flap.

14. The securement device of claim 13, wherein the first fastener and the flap are outwardly facing.

15. The securement device of claim 13, wherein the fabric retaining member is band made of a stretchable material such that a size of the band is adjustable.

16. The securement device of claim 13, further comprising a second fastener operably connected to the flap, the second fastener configured to releasably couple the flap with the base member proximate to the first fastener.

17. The securement device of claim 16, wherein the second fastener comprises:
a second fastener component disposed on the base member; and
another second fastener component disposed at the distal end of the flap and configured to engage the second fastener component disposed on the base member to mutually, releasably couple the flap with the base member.

18. The securement device of claim 16, wherein at least one of the first fastener or the second fastener comprises a snap, a hook-and-loop fastener, a magnet, or a combination thereof.

19. A securement device for medical tubing, comprising:
a base member comprising a proximal end and a distal end that includes a first fastener component, wherein the base member is substantially free of adhesive and further includes a second fastener component disposed proximate to the first fastener component;
a flap operably connected to the base member, the flap and base member together defining an opening configured to accommodate medical tubing, wherein the flap comprises a proximal end affixed to the base member and a distal end that is selectively detachable from the base member, wherein the flap includes a second fastener component at the distal end of the flap; and
a fabric retaining member operably connected to the base member, the fabric retaining member comprising a distal end that is affixed to the base member and a proximal end that includes a first fastener component;
wherein the first fastener component of the fabric retaining member is configured to engage the first fastener component of the base member to form a first fastener, wherein the first fastener is configured to mutually, releasably couple the fabric retaining member with the base member proximate to the distal end of the flap; and
wherein the second fastener component of the flap is configured to engage the second fastener component of the base member to form a second fastener, wherein the second fastener is configured to mutually, releasably couple the flap with the base member proximate to the first fastener.

20. The securement device of claim 19, wherein the base member includes a platform that extends upward from the base member, the platform comprising a top side that is substantially flat and is configured to support the flap when the flap is releasably coupled with the base member.
